(19) United States
(12) Patent Application Publication
(10) Pub. No.: US 2005/0142182 A1
(43) Pub. Date: Jun. 30, 2005

(54) Long circulating liposome

(75) Inventors: Ae-June Wang, Hsinchu City (TW); Pei-Lin Wang, Chiai City (TW); Shin-Jr Lu, Niausung Shiang (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Chutung (TW)

(21) Appl. No.: 11/023,525

(22) Filed: Dec. 29, 2004

(30) Foreign Application Priority Data
Dec. 31, 2003 (TW) 092137776

(51) Int. Cl. A61K 9/127; C12N 15/88

(52) U.S. Cl. 424/450; 435/458

(57) ABSTRACT

The present invention relates to a liposome having a phospholipid bilayer and a hydrophilic core, wherein the phopspholipid bilayer contains D-alpha tocopheryl polyethylene glycol 1000 succinate (TPGS). The liposomes are first prepared by solvent injection and extrusion method, and then drug loading by ammonium sulfate gradient. The TPGS in the liposome composition can prolong the circulation time of liposomes and thus increase the chance for the drug composition to enter target sites so as to improve the efficiency of drug delivery.

### BACKGROUND OF THE INVENTION

1. Field of the Invention

The present invention relates to a novel liposome, more particularly, to a liposome suitable for prolonging the circulation time in a subject's body.

2. Description of Related Art

Liposomes have various functions, according to the design, structure and size. Traditional liposomes mainly consist of phospholipid and cholesterol with neutral or negative charge. Disregard of the surface charge, lipid ingredients or particle size, these liposomes all have a main feature—the circulation time in a human body is very short. The reason for this is the liposome will be catched by macrophages of the immune system to release a drug immediately after injection, and macrophages are often located in the reticuloendothelial system (RES) consisting of liver, spleen, brain, lymph nodes and lung. In addition, liposomes are mainly located in liver and spleen where abundant blood cells and macrophages exist. Therefore, such liposomes are suitable for carrying some immunoregulatory medicines, such as vaccine, or anti-infective drug, such as liposomal hepatitis-A vaccine which has been available in the Swiss pharmaceutical market since 1994. Another anti-infective drug, Amphotericin B™, is also such type of drug.

At the end of the 1980s, the landmark of the liposome development was the invention of long-circulating liposomes. Such improved liposomes are prepared by modifying the surface molecules of traditional liposomes, i.e. coupling with hydrophilic polymers such as polyethylene glycol to form a stable 3-D structure. Such structure can avoid being recognized and eliminated by immune system and thus prolong the circulation time of liposomes in the body. Furthermore, the long circulating liposomes could extravasates selectively into specific tissues (ex. tumor) through the leakages occurred in tumor neovasculature. Therefore, such liposomes are thus aggregated in the specific tissues such as cancer cells, inflamed and infected tissues to release the drugs and achieve targeted-specific drug delivery. The particle size of such liposome is approximately 100 nm. For traditional drugs, only approximately 1% of initial drugs will reach the target site after intravenous injection; however, for the drugs encapsulated in long-circulating liposomes, the circulation time can be prolonged to 24 hours or even up to 48 hours. This suggests that about more than 1% (even up to 10%) of initial drugs in blood will accumulated near by the specific tissues, and the efficacy is consequently highly increased. Such a representative product is Doxil™, developed by Sequus Co., which comprises Doxorubicin™, an anti-cancer drug, coated with PEG liposomes (Stealth). This drug has been available on the market since 1995 and is used to treat cancer such as Kaposi sarcoma for AIDS patients, ovarian cancer and breast cancer patients. There are still several other treatments for cancer under clinical trial.

The derivatives of D-α-tocopherol are known to be used as solutizers which help specific drugs to be delivered into a body. For example, amphiphilic vitamin E(D-alpha tocopheryl polyethylene glycol 1000 succinate, TPGS) has been used as a solutizer of Paclitaxel™ or as other cosmetic ingredients.

### SUMMARY OF THE INVENTION

It is the object of the present invention to provide a novel liposome composition which can prolong the circulation time of liposomes in the blood and enhance the delivery efficiency of drugs to the target tissues. The damage to normal cells and side effects are thus eliminated, and the efficacy is thus increased.

To achieve the object, the present invention provides a liposome, comprising a phospholipid bilayer structure and a hydrophilic core, wherein the essential lipids and a key component—amphiphilic vitamin E (D-alpha tocopheryl polyethylene glycol 1000 succinate, TPGS).

TPGS is prepared from the esterification of D-α tocopheryl acid succinate and polyethyleneglycol 1000. TPGS is a amphiphilic vitamin E, very stable under normal conditions without hydrolysis. Because of its HLB (hydrophile-lipophile balance) value being between 15 and 19, TPGS has excellent water solubility and is also suitable to serve as a surfactant which can emulsify hydrophobic drugs. Therefore, if TPGS is added to the composition of liposomes or microemulsion, the stability of liposomes could be highly increased. In addition, the composition is even more suitable for specific target drug delivery in the body, as it can prevent liposomes from being evacuated by immune systems so as to prolong the circulation time of liposomes in the blood to reach the targeted tissue. The damage to normal cells and side effects are eliminated, and the efficacy is accordingly increased.

### BRIEF DESCRIPTION OF THE DRAWINGS

FIG. 1 is a plasma concentration versus time diagram for Doxorubicin of Example 3.

### DETAILED DESCRIPTION OF THE PREFERRED EMBODIMENT

### Example 1

### Preparation of Liposomes Containing TPGS and DCP

The liposomes containing TPGS and DCP are prepared according to the formulation listed in Table 1 by the following processes.

TABLE 1
Formulation of liposomes in Example 1

| | | HSPC | Cholesterol | TPGS | Vitamin E | DCP |
|---|---|---|---|---|---|---|
| Initial | | 3 | 1.5 | 0.75 | 0.43 | 0.3 |
| (weight | | | | | | |
| ratio) | | | | | | |
| Weight | [mg] | 9.58 | 4.79 | 2.4 | 1.37 | 0.96 |
| Volume | ×3 | 28.74 | 14.37 | 7.19 | 4.12 | 2.87 |
| Increased | ×3 | 86.22 | 43.11 | 21.56 | 12.36 | 8.62 |
| initial | | | | | | |
| conc. | | | | | | |

86.22 mg of hydrogenated soy phosphatidyl choline (HSPC), 43.11 mg of cholesterol, 21.56 mg of TPGS, 12.36 mg of vitamin E, and 8.62 mg of dicetylphosphate (DCP) are weighed in vials and added with 0.3 ml of ethanol. The mixture is heated in 60° C. water bath to dissolve all solutes in ethanol.

The solution is then injected to 2.7 ml of 250 mM (NH₄)₂SO₄ in an isothermal circulation beaker and stirred to perform hydration for 1 hour.

After completion of hydration, the particle extrusion process is performed, and multi-lamellar vesicles (MVLs) are sieved by filter membranes with aperture of 0.1 μm, and 0.05 μm respectively to obtain small unilamellar vesicles (SUVs).

SUV liposome solutions are then poured into a pretreated dialysis tube, and the first dialysis is performed in 250 mM (NH₄)₂SO₄ solution for 8 hours. The second dialysis is then performed in a solution containing 10% of sucrose and 5 mM of NaCl until the solution surrounding SUV liposomes contains no (NH₄)₂SO₄.

The amount and concentration of phospholipids are determined by the Bartlett assay method.

After determining the concentration, SUV liposomes and Doxorubicin are mixed with a weight ratio of 34 mg/ml:4 mg/ml (the volume ratio of liposomes to Doxorubicin is 1:1) in a 60° C. water-bath for 1 hour to perform drug-loading and Doxorubicin-containing liposomes are then obtained.

### Example 2

### Preparation of Liposomes Containing TPGS and Vitamin E

The liposomes containing TPGS and distearoylphosphatidylethanolamine Methoxy-poly(ethylene) glycol (DSPE-MPEG) are prepared according the formulation listed in Table 2 by the following processes.

TABLE 2
Formulation of liposomes of Example 2

| | | | | | DSPE-M | |
|---|---|---|---|---|---|---|
| | | HSPC | Cholesterol | TPGS | PEG | Vitamin E |
| Initial | | 3 | 1.5 | 0.3 | 1 | 0.5 |
| (weight | | | | | | |
| ratio) | | | | | | |
| Weight | [mg] | 9.58 | 4.79 | 0.96 | 3.19 | 1.6 |
| Volume | ×3 | 28.74 | 14.37 | 2.87 | 9.58 | 4.79 |
| part | | | | | | |
| Increase | ×3 | 86.22 | 43.11 | 8.62 | 28.74 | 14.37 |
| initial | | | | | | |
| conc. | | | | | | |

86.22 mg of hydrogenated soy phosphatidyl choline (HSPC), 43.11 mg of cholesterol, 21.56 mg of TPGS, 12.36 mg of Vitamin E, and 8.62 mg of dicetylphosphate (DCP) are weighed in vials and added with 0.3 ml of ethanol. The mixture is heated in a 60° C. water bath to dissolve all samples in ethanol.

The solution is then injected to 2.7 ml of 250 mM (NH₄)₂SO₄ in an isothermal circulation beaker and stirred to perform hydration for 1 hour.

After completion of hydration, the particle extrusion process is performed, and MVLs are sieved by filter membranes with the pore size of 0.1 μm, and 0.05 μm respectively to obtain SUVs.

SUV liposome solutions are poured into a pretreated dialysis tube, and the first dialysis is performed in 250 mM (NH₄)₂SO₄ solution for 8 hours. The second dialysis is then performed in a solution containing 10% of sucrose and 5 mM of NaCl until the solution surrounding SUV liposomes contains no (NH₄)₂SO₄.

The amount and concentration of phospholipid are determined by the Bartlett assay method.

After determining the concentration, SUV liposomes and Doxorubicin are mixed with weight ratio of 34 mg/mL:4 mg/mL (the volume ratio of liposomes to Doxorubicin is 1:1) in 60° C. water-bath for 1 hour to perform drug-loading and Doxorubicin-containing liposomes are then obtained.

### Example 3

### The Circulation Time of the Liposomes of the Present Invention in the Body

In this example, the concentration of Doxorubicin in the blood is determined by a pharmacokinetics method, and HPLC analysis is performed subsequently.

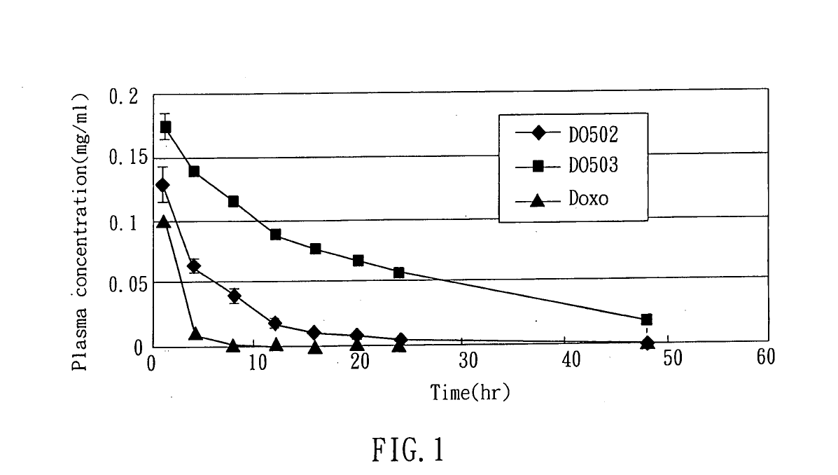

Doxo. is compared with DO503 liposomes (Example 1) and DO502 liposomes (Example 2), wherein Doxo. represents the free doxorubicin solution. With reference to FIG. 1, there is almost no Doxorubicin concentration detected in rat blood 8 hours after injection; on the contrary, DO502 and DO503 liposomes still exist in the rat blood 24 hours after injection. The results suggest that the liposome composition containing TPGS of the present invention possesses a long-circulating property. Especially for DO503, it even exists in rat blood 48 hours after injection. The results show that the liposomes containing TPGS can prolong the circulation time and hence increase the drug potency.

It is noticeable that pharmaceutical ingredient encapsulated in liposome of the present invention is doxorubicin; however, the preferred pharmaceutical ingredients are selected, but not limited to, from a group consisting of viruses, vectors, proteins, peptides, nucleic acids, polysaccharides, carbohydrates, lipids, glycoproteins, pharmaceutical ingredients and the mixture thereof. The phospholipid bilayer of the liposomes of the present invention may be a well-known phospholipid added with TPGS, and preferably consisting of TPGS, HSPC, cholesterol, DCP and vitamin E; or consisting of TPGS, HSPC, cholesterol, DSPE-MPEG and vitamin E. The amount of TPGS contained in liposomes is preferably 4 wt % to 35 wt % (weight ratio of solute). The amount of DCP contained in liposomes is preferably 1 wt % to 14 wt % (weight ratio of solute). The amount of DSPE-MPEG contained in liposomes is preferably 5 wt % to 20 wt % (weight ratio of solute). The phospholipids suitable liposomes of the present invention are, but not limited to, saturated or unsaturated phosphatidyl choline, such as hydrogenated natural phospholipids or long chain saturated phospholipids, unsaturated phospholipid or short chain saturated phospholipids. Long chain saturated phospholipids are preferably phosphatidyl choline (PC), phosphatidyl glycerol (PG), phosphatidyl serine (PS) or phosphatidyl ethanolamine (PE). Phosphatidyl choline is preferably, but not limited to, hydrogenated egg phosphatidyl choline (HEPC) or hydrogenated soy phosphatidyl choline (HSPC). Long chain saturated phosphatidyl choline is preferably, but not limited to, dipalmitoyl phosphatidyl choline (DPPC), distearyloyl phosphatidyl choline (DSPC) or the mixture thereof. Unsaturated phosphatidyl choline is preferably, but not limited to, egg phosphatidyl choline (EPC), soy phosphatidyl choline (SPC), other artificial unsaturated PCs or natural unsaturated PC. Short chain saturated phosphatidyl choline is preferably, but not limited to, dilauroyl phosphatidyl choline (DLPC).

Although the present invention has been explained in relation to its preferred embodiment, it is to be understood that many other possible modifications and variations can be made without departing from the spirit and scope of the invention as hereinafter claimed.

1. A liposome, comprising a phospholipid bilayer and a hydrophilic core, wherein the phospholipid bilayer contains vitamin E derivative (D-alpha tocopheryl polyethylene glycol 1000 succinate, TPGS).

2. The liposome as claimed in claim 1, wherein the hydrophilic core further comprises at least one bioactive ingredient.

3. The liposome as claimed in claim 2, wherein said at least one bioactive ingredient is selected from the group consisting of viruses, vectors, proteins, peptides, nucleic acids, polysaccharides, carbohydrates, lipids, glycoproteins and pharmaceutical ingredients.

4. The liposome as claimed in claim 1, wherein the phospholipid bilayer comprises vitamin E derivative (D-alpha tocopheryl polyethylene glycol 1000 succinate, TPGS), hydrogenated soybean phosphatidyl choline (HSPC), cholesterol, dicetylphosphate (DCP) and vitamin E.

5. The liposome as claimed in claim 1, wherein the phospholipid bilayer comprises vitamin E derivative (D-alpha tocopheryl polyethylene glycol 1000 succinate, TPGS), hydrogenated soybean phosphatidyl choline (HSPC), cholesterol, distearoyl-phosphatidyl ethanolamine methoxy-polyglycerol (DSPE-MPEG) and vitamin E.

6. The liposome as claimed in claim 4, wherein the liposome comprises 4 wt % to 35 wt % of hydrophilic vitamin E (D-alpha tocopheryl polyethylene glycol 1000 succinate, TPGS).

7. The liposome as claimed in claim 4, wherein the liposome comprises 1 wt % to 14 wt % of dicetylphosphate (DCP).

8. The liposome as claimed in claim 5, wherein the liposome comprises 4 wt % to 35 wt % of vitamin E derivative (D-alpha tocopheryl polyethylene glycol 1000 succinate, TPGS).

9. The liposome as claimed in claim 5, wherein the liposome comprises 5 wt % to 20 wt % of DSPE-MPEG.

10. A drug delivery system, comprising at least one liposome, wherein the liposome comprises a phospholipid bilayer and a hydrophilic core, wherein the phospholipid bilayer contains vitamin E derivative (D-alpha tocopheryl polyethylene glycol 1000 succinate, TPGS).

11. The drug delivery system as claimed in claim 10, wherein the hydrophilic core further comprises at least one bioactive ingredient.

12. The drug delivery system as claimed in claim 11, wherein said at least one bioactive ingredient is selected from the group consisting of viruses, vectors, proteins, peptides, nucleic acids, polysaccharides, carbohydrates, lipids, glycoproteins, pharmaceutical ingredients and the mixture thereof.

13. The drug delivery system as claimed in claim 10, wherein the phospholipid bilayer comprises vitamin E derivative (D-alpha tocopheryl polyethylene glycol 1000 succinate, TPGS), hydrogenated soybean phosphatidyl choline (HSPC), cholesterol, dicetylphosphate (DCP) and vitamin E.

14. The drug delivery system as claimed in claim 10, wherein the phospholipid bilayer comprises vitamin E derivative (D-alpha tocopheryl polyethylene glycol 1000 succinate, TPGS), hydrogenated soybean phosphatidyl choline (HSPC), cholesterol, distearoyl phosphatidyl ethanolamine-methoxy polyglycerol (DSPE-MPEG) and vitamin E.

15. The drug delivery system as claimed in claim 13, wherein the liposome comprises 4 wt % to 35 wt % of vitamin E derivative (D-alpha tocopheryl polyethylene glycol 1000 succinate, TPGS).

16. The drug delivery system as claimed in claim 13, wherein the liposome comprises 1 wt % to 14 wt % of dicetylphosphate (DCP).

17. The drug delivery system as claimed in claim 14, wherein the liposome comprises 4 wt % to 35 wt % of vitamin E derivative (D-alpha tocopheryl polyethylene glycol 1000 succinate, TPGS).

18. The drug delivery system as claimed in claim 14, wherein the liposome comprises 5 wt % to 20 wt % of dicetylphosphate (DCP).
